(19) United States
(12) Plant Patent Application Publication
(10) Pub. No.: US 2013/0160174 P1
(43) Pub. Date: Jun. 20, 2013

(54) Helleborus plant named 'ABCRD01'

(50) ;

(71) Applicants: Rodney Davey, Devon (GB); Lynda Christine Windsor, Devon (GB)

(72) Inventors: Rodney Davey, Devon (GB); Lynda Christine Windsor, Devon (GB)

(73) Assignee: AMAZING BREEDERS COMPANY B.V., Aalsmeer (NL)

(21) Appl. No.: 13/374,191

(22) Filed: Dec. 15, 2011

(51) Int. Cl. A01H 5/00

(52) U.S. Cl. PLT/439

(57) ABSTRACT

A new Helleborus plant particularly distinguished by abundant red-purple flowers, upright plant habit and a vigorous growth habit, is disclosed.

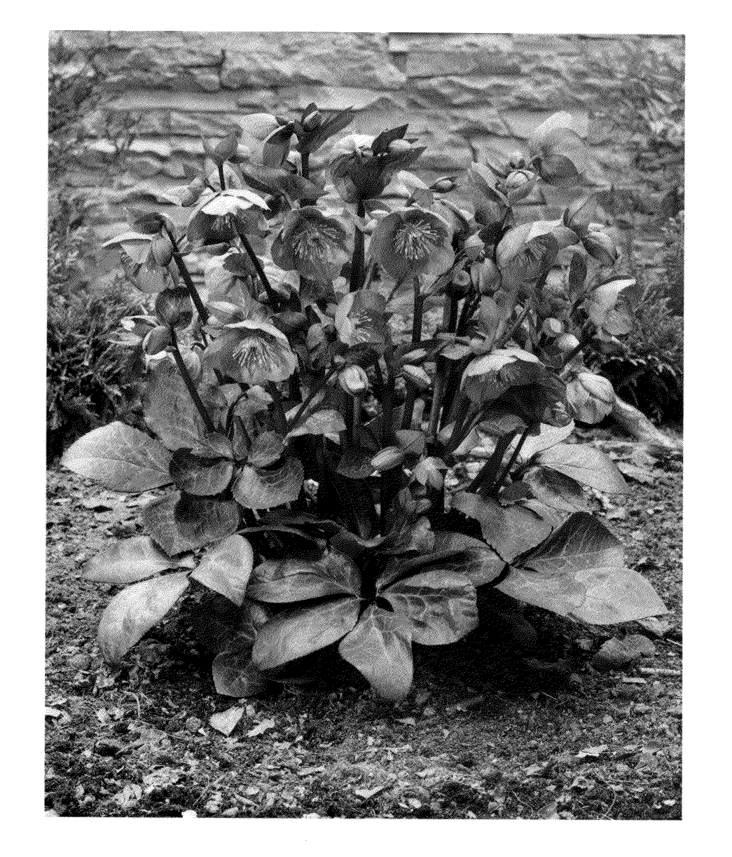

### GENUS AND SPECIES

Helleborus×hybridus

### VARIETY DENOMINATION

‘ABCRD01’

### BACKGROUND OF THE NEW PLANT

The present invention comprises a new and distinct variety botanically known as Helleborus×hybridus, and hereinafter referred to by the variety name ‘ABCRD01’. The new variety originated from a controlled hybridization conducted in February 2007 in Devon, United Kingdom, between female hellebore plant Helleborus nr. 3469RDEX2 (unpatented) and male hellebore plant Helleborus nr. 3465RDMT45 (unpatented). The seeds produced by the hybridization were sown in Devon, United Kingdom at RD Plants Company. A single plant selection was chosen for further evaluation and for asexual propagation in Devon, United Kingdom.

‘ABCRD01’ was subsequently asexually propagated by tissue culture in Aalsmeer, The Netherlands for 3 years. The present invention has been found to retain its distinctive characteristics through successive asexual propagations for at least 50 generations.

Community Plant Variety Rights (CPVR) for this variety were applied for in Europe on Oct. 25, 2011. ‘ABCRD01’ has not been made publicly available more than one year prior to filing of this application.

### SUMMARY OF THE INVENTION

The following are the most outstanding and distinguishing characteristics of this new variety when grown under normal horticultural practices in Aalsmeer and Boijl, The Netherlands.

1. Abundant, red-purple flowers;

2. Upright plant habit; and

3. A vigorous growth habit.

### DESCRIPTION OF THE PHOTOGRAPH

This Helleborus plant is illustrated by the accompanying photograph which shows overall plant habit including blooms, buds, and foliage of the plant; the colors shown are as true as can be reasonably obtained by conventional photographic procedures. The photograph is of a whole plant about one-year-old, grown outdoors in Boijl, The Netherlands.

### DESCRIPTION OF THE NEW VARIETY

The following detailed description sets forth the distinctive characteristics of ‘ABCRD01’. The data which define these characteristics were collected from asexual reproductions carried out in Aalsmeer and Boijl, The Netherlands. The plant history was taken on one-year-old plants grown outdoors. Color readings were taken under artificial light. Color references are primarily to the R.H.S. Color Chart of The Royal Horticultural Society of London (R.H.S.) (2001).

### DETAILED BOTANICAL DESCRIPTION

Classification:
   Family.—Ranunculaceae.
   Genus and species.—Helleborus×hybridus.
   Common name.—Christmas Rose.
   Variety name.—‘ABCRD01’.
Plant:
   Plant and growth habit.—Upright and vigorous.
   Propagation.—In vitro propagation by tissue culture.
   Height.—25.0 cm to 35.0 cm.
   Width.—20.0 cm to 25.0 cm.
   Time to initiate roots.—6 weeks.
   Time to produce a rooted cutting.—Approximately 3 months.
   Time to produce a finished plant.—3 months.
   Root habit.—Vigorous and fleshy roots.
Stems:
   Shape.—Compact.
   Length.—3.0 cm.
   Diameter.—2.0 cm.
   Internode length.—0.1 cm.
   Texture.—Smooth.
   Color.—RHS 183C.
Leaves:
   Type.—Palmately compound.
   Arrangement.—Alternate.
   Length.—15.0 cm to 20.0 cm.
   Width.—15.0 cm to 20.0 cm.
   Shape.—Ovate.
   Apex.—Obtuse.
   Base.—Acute.
   Margin.—Serrate.
   Texture (both upper and lower surfaces).—Smooth.
   Color (both immature and mature leaves).—Upper surface: RHS 139A Lower surface: RHS 138A.
   Venation pattern.—Longitudinal.
   Venation color (both upper and lower surfaces).—RHS 138A.
   Venation color (at base of leaf).—Mature leaf: RHS 187A Immature leaf: RHS 59B.
   Variegation.—Absent.
   Leaflets.—Quantity per leaf: 4 to 6 Shape: More cordate than oval Length: 6.0 cm to 10.0 cm, depending on the growing stage Width: 4.0 cm to 6.0 cm, depending on the growing stage Apex: Acute Base: Between cordate and obtuse Margin: Serrate.
   Petiole.—Length: 20.0 cm to 25.0 cm Diameter: 0.5 cm Color: RHS 183C Texture: Smooth.
   Petiolule.—Length: 2.0 cm Diameter: 0.5 cm Color: RHS 183C Texture: Smooth.
   Stipules.—Absent.
Inflorescence:
   Type.—Loosely branched cymes.
   Shape.—Loosely branched.
   Self cleaning or persistent.—Persistent.
   Flowering habit.—Vertical facing flowers above the foliage.
   Flowering season.—January through February.
   Quantity of inflorescences on the plant.—10 to 15.
   Fragrance.—Absent.
   Lastingness of inflorescences on the plant.—At least 2 months.
   Diameter.—15.0 cm.
   Depth (height).—30.0 cm.
Flower:
   Type.—Single.
   Diameter.—4.0 cm to 5.0 cm.
   Depth (height).—0.8 cm to 1.0 cm.
   Quantity per inflorescence.—2 to 3.
Bud:
   Height.—3.0 cm.
   Diameter.—2.0 cm.
   Shape.—Ovate.
   Colour.—RHS 70B.
   Texture.—Smooth.
Bracts:
   Shape.—Ovate.
   Quantity per inflorescence.—2 to 10.
   Length.—3.0 cm.
   Width.—2.0 cm.
   Apex.—Acute.
   Base.—Ovate.
   Margin.—Smooth.
   Texture (both upper and lower surfaces).—Smooth.
   Color.—Upper surface: RHS 139A Lower surface: RHS 138A.
Peduncle:
   Shape.—Round.
   Length.—20.0 cm.
   Width.—0.5 cm.
   Texture.—Smooth.
   Color.—RHS 183B.
Pedicel:
   Length.—2.0 cm to 5.0 cm.
   Diameter.—0.3 cm.
   Texture.—Smooth.
   Color.—RHS 183B.
Sepals:
   Quantity per flower.—5.
   Arrangement.—Single whorl.
   Shape.—Orbicular.
   Length.—2.5 cm.
   Width.—2.5 cm.
   Apex.—Rounded.
   Base.—Rounded.
   Margin.—Smooth.
   Texture (both upper and lower surfaces).—Smooth.
   Color.—Upper surface: RHS 70B Lower surface: RHS 70C.
Reproductive organs:
   Stamens.—Quantity per flower: Greater than 60 Color: RHS 4D Filament length: 1.5 cm Filament width: 0.05 cm.
   Anthers.—Shape: Ovate Length: 0.2 cm Diameter: 0.1 cm Color: RHS 4B Pollen amount: Sparse Pollen color: RHS 4B.
   Pistils.—Quantity per flower: 5 Shape: Tuberculate Length: 1.5 cm Width: 0.1 cm Color: RHS 183A.
   Stigma.—Length: 0.2 cm Width: 0.05 cm Color: RHS 157C.
   Style.—Length: 0.5 cm to 0.7 cm Color: RHS 71C.
   Ovary.—Shape: Ovate Color: RHS 139A.
   Nectaries.—Quantity per flower: 10 to 14 Shape: Funnel Length: 0.5 cm to 0.7 cm Width: 0.2 cm to 0.3 cm Color: RHS N144B.
Fruit and seed set: None observed
Disease and insect resistance: No particular resistance or susceptibility observed
Physiological characteristics: Tolerant to frost

### COMPARISON WITH COMMERCIAL VARIETIES

‘ABCRD01’ differs from the commercial variety ‘HGP01’ (U.S. Plant Pat. No. 21,062) in that ‘ABCRD01’ has a flower bud color of RHS 70B (red-purple), whereas ‘HGP01’ has a flower bud color close to RHS 184C (grayed-purple). Additionally, ‘ABCRD01’ has red-purple sepals (RHS 70B), whereas ‘HGP01’ has white sepals (RHS 155C) that become closer to pink (RHS 56D) with development.

We claim:

1. A new and distinct variety of Helleborus plant as described and shown herein.
